(19) United States
(12) Patent Application Publication
(10) Pub. No.: US 2026/0060557 A1
(43) Pub. Date: Mar. 5, 2026

(54) SENSOR HEAD DEVICE FOR A MINIMAL INVASIVE VENTRICULAR ASSIST DEVICE AND METHOD FOR PRODUCING SUCH A SENSOR HEAD DEVICE

(71) Applicant: KARDION GMBH, Stuttgart (DE)

(72) Inventors: Thomas Alexander Schlebusch, Renningen (DE); Julian Kassel, Böblingen (DE)

(21) Appl. No.: 19/384,914

(22) Filed: Nov. 10, 2025

Related U.S. Application Data
(63) Continuation of application No. 15/734,036, now Pat. No. 12,478,267.

(30) Foreign Application Priority Data
Jun. 6, 2018 (DE) 102018208892.6

(51) Int. Cl. A61B 5/0215; A61B 5/00; A61B 5/01; A61B 5/026

(52) U.S. Cl. CPC A61B 5/02158; A61B 5/01; A61B 5/026; A61B 5/6852; A61B 5/686; A61B 2562/0247; A61B 2562/0271; A61B 2562/06; A61B 2562/12

(57) ABSTRACT

The invention relates to a sensor head device for a heart support system, wherein the sensor head device has at least one sensor carrying element, wherein the sensor carrying element has at least one sensor cavity for accommodating at least one sensor and/or at least one signal transmitter cavity for accommodating at least one signal transmitter.

### INCORPORATION BY REFERENCE TO ANY PRIORITY APPLICATIONS

The present application is a continuation of U.S. application Ser. No. 15/734,036 filed on Jun. 8, 2021, which is a National State entry of PCT Patent Application No. PCT/EP2019/064780, filed on Jun. 6, 2019, titled SENSOR HEAD DEVICE FOR A MINIMALLY INVASIVE HEART SUPPORT SYSTEM AND METHOD FOR PRODUCING SUCH A SENSOR HEAD DEVICE, which is an International Application of and claims the benefit of priority to German Patent Application No. 102018208892.6, filed on Jun. 6, 2018, the contents of each of which are hereby incorporated by reference herein in their entirety as if fully set forth herein for all purposes. Any and all applications for which a foreign or domestic priority claim is identified in the Application Data Sheet as filed with the present application are hereby incorporated by reference under 37 CFR 1.57.

### BACKGROUND

### Field

The invention proceeds from a device or a method according to the class of the independent claims. The subject matter of the present invention is also a computer program.

### Description of the Related Art

Heart support systems, such as implanted left ventricular support systems, can have a plurality of sensors for detecting body parameters.

Proceeding therefrom, the invention is based on the object of improving a sensor head device in the sense of an optimization of the sensor or signal transmitter integration and encapsulation and to specify a corresponding advantageous production method.

### SUMMARY

Against this background, a sensor head device for a minimally invasive heart support system, a method for producing a sensor head device for a heart support system, and a device that uses this method as well as, lastly, a corresponding computer program according to the main claims are presented with the approach presented here. Advantageous developments and improvements of the device specified in the independent claim are possible by means of the measures listed in the dependent claims.

The approach presented here of a sensor head device for a heart support system relates to improved integration and encapsulation of sensor technology in a, for example, minimally invasive left ventricular heart support system in aortic valve position. In this case, the sensor head device is designed as a plug-shaped sensor package, for example, which can, for example, be arranged at a distal end of a supply cannula of a heart support system, and has at least one cavity for at least one electronic sensor and/or for at least one signal transmitter, e.g., an ultrasound element.

A sensor head device for a minimally invasive heart support system insertable into a patient is presented, wherein the sensor head device has at least the following feature:
a sensor carrying element having at least one sensor cavity for accommodating at least one sensor and/or at least one signal transmitter cavity for accommodating at least one signal transmitter.

A sensor head device can be a structural element of a heart support system, which serves to integrate or accommodate a sensor assembly, e.g., a temperature sensor and/or pressure sensor and/or an ultrasound element. A heart support system, also called artificial heart or VAD (ventricular assist device), can be understood to mean a pumping device for increasing the pump performance of a heart. The heart support system can be insertable into a heart chamber or into the aorta by means of a catheter, for example. The heart support system may in particular be a (percutaneous) left ventricular support system. A sensor carrying element can be a carrying component, e.g., a housing, a rack, and/or a frame, on which at least one sensor and/or at least one signal transmitter can be or is arranged, wherein the sensor and/or the signal transmitter is mechanically protected and securely supported by the sensor carrying element. A sensor cavity can be understood to mean a cavity (e.g., formed squarely or cubically or in the form of an at least partially circumferential groove), which is formed, for example, in a sensor carrying element of a sensor head device and serves to accommodate and encapsulate a sensor. A sensor can, for example, be a technical component which can detect specific physical variables, such as temperature and pressure, either qualitatively or quantitatively as a measured variable. These variables can in this case be detected by means of physical effects and, for example, converted into a further processable electrical signal. A signal transmitter cavity can be understood to mean a cavity (e.g., formed squarely or cylindrically) that is formed, for example, in a sensor carrying element of a sensor head device and serves to accommodate and encapsulate a signal transmitter. A signal transmitter can, for example, be an ultrasound element as a transmitter and/or receiver for ultrasound.

The advantages of the approach presented here of a sensor head device for a heart support system (e.g., a heart support system that is minimally invasively insertable into a patient) in particular consist in creating a possibility for optimized integration and encapsulation of at least one pressure sensor and/or temperature sensor in a tip of a heart support system. There are currently no systems available on the market that insert active electronic components and sensor technologies in the head region of a heart support system. In this case, a blood temperature of a person with heart disease can advantageously be measured by an integrated temperature sensor, for example. Since sepsis is a common complication in patients with an implanted heart support system, a febrile infection can be detected in good time and treated or prevented early on by means of a trend analysis of the blood temperature, for example. By means of improved integration and encapsulation of a (preferably barometric) pressure sensor, a ventricular pressure of a heart disease patient can be detected, for example. The integration in this case is designed, for example, in such a way that the pressure measurement is not falsified by the slight negative pressure in a guide cannula of the heart support system. The determined pressure data can be used, for example, to measure the ventricular pressure of a patient and to regulate the pump on the basis of the ventricular pressure in order to protect the ventricle from collapse, for example. The pressure data determined in this way can also be used, for example, to determine a differential pressure, i.e., aortic pressure minus ventricular pressure, across the pump and thus to monitor the pump, the pump performance, or the function of the pump.

According to one embodiment, the sensor carrying element can have a channel extending in the longitudinal direction of extension of the sensor head device and/or the at least one sensor cavity can be open in a different direction than the at least one signal transmitter cavity. Such an embodiment of the approach presented here offers the advantage that such a channel allows a guide wire to be guided centrally through the sensor carrier provided with sensor cavities. This ensures optimum mechanical protection and secure mounting. A very precise measurement of body parameters can also be achieved as a result of the different opening directions of the cavities.

According to one embodiment, the sensor carrying element can have a rounded cap at one outer end. In cross section, the rotationally symmetric sensor carrying element can alternatively or additionally have outer walls (e.g., cylindrical outer walls) arranged in the shape of a U and/or E and/or have inner walls (e.g., cylindrical inner walls) arranged in the shape of a U and/or E at at least one inner end, wherein the at least one signal transmitter cavity or sensor cavity is in particular formed between the outer walls and/or between the outer and inner walls. Such an embodiment of the approach presented here offers the advantage that an outer end with a rounded cap or an outer end of the sensor head device designed in the shape of a mushroom head is advantageously formed to introduce the heart support system into the patient in order to not induce any injuries in a heart disease patient or to ensure good sliding properties, for example, when the heart support system is advanced during implantation of the system. In this case, the outer end designed in the shape of a mushroom head, but also, for example, an outer end designed in the shape of a pot, continues to provide mechanical protection and secure encapsulation for the integrated sensors.

Such an embodiment of the approach presented here furthermore offers the advantage that an embodiment of the sensor head device as a type of stopper at the end of a guide cannula of a heart support system allows simple production of the sensor head device, wherein the manufacturing process saves time and money.

According to one embodiment, the sensor carrying element can have an opening in its center, wherein the opening has in particular a cylindrical tube. The tube can be inserted into the opening and/or formed therein over the entire length of the sensor carrying part, for example. Such an embodiment of the approach presented here offers the advantage that a guide wire can, for example, be inserted into the opening and/or into the cylindrical tube. When implanting a heart support system into the left heart chamber or the aorta of a heart disease patient, the guide wire can first be laid to the ventricle of the patient. The heart support system can then be pushed onto the guide wire and advanced along the guide wire to the end position. In this way, very precise positioning of the heart support system can be achieved.

According to one embodiment, the sensor head device can have at least one sensor, in particular a sensor designed as a temperature sensor and/or pressure sensor, and/or at least one signal transmitter, in particular a signal transmitter designed as an ultrasound element or ultrasound transducer for emitting and/or receiving ultrasound. Alternatively or additionally, the at least one sensor and/or the at least one signal transmitter can be arranged on an electrical conductive element. The ultrasound element can, for example, also have its own housing, wherein an outer wall around the ultrasound element can be omitted. In this case, the wall can be used for both mechanical stabilization and encapsulation of the ultrasound element for the purpose of biocompatibility. Such an embodiment of the approach presented here offers the advantage that integration of at least one ultrasound element into a sensor head device of a heart support system allows a volume flow measurement of the blood of the heart disease patient.

According to one embodiment, the signal transmitter cavity can be covered at least partially by a lens element, wherein the lens element is in particular designed as an ultrasonic lens and/or made of a silicone material. Such an embodiment of the approach presented here offers the advantage that an ultrasonic lens is used for sound formation of the ultrasonic energy, the ultrasonic lens furthermore securely encapsulates the ultrasound element, and the ultrasonic lens can conduct the blood flow of the heart disease patient entering through a plurality of inlet ports.

According to one embodiment, the sensor carrying element can be formed from a thermoplastic and/or made in a machining manufacturing process and/or as an injection molded part. Such an embodiment of the approach presented here offers the advantage that high-quality and resilient medical products can be manufactured by means of injection molding processes, for example. The flush production from the original raw material to the finished injection molded part makes the injection molding process a fast and cost-effective process, which can be used, for example, for the manufacturing of precision parts. Particularly extremely small individual parts, such as those for the manufacture of a minimally invasive heart support system, benefit from the exact production. As a rule, further post-processing of the finished part is not necessary so that production can be completed quickly.

According to one embodiment, the sensor cavity can be filled with a casting compound, wherein the casting compound in particular comprises a solid or gel-like silicone or a silicone oil. Such an embodiment of the approach presented here offers the advantage that filling the sensor cavity with a casting compound can protect the sensors from blood and mechanical damage, for example. In this case, the casting compound should be able to transfer the cardiac pressure of the patient determined by a pressure sensor in order to continue to enable precise pressure measurements. The casting compound can be, for example, a solid and/or gel-like silicone or even a silicone oil, wherein this casting compound necessitates an additional encapsulation of the sensors.

According to one embodiment, the sensor cavity can be coated and/or covered by means of a membrane and/or a diffusion barrier, wherein the membrane is in particular designed as a film and/or a layer separated from a gas or vapor phase and/or the diffusion barrier comprises a parylene material. When using a silicone oil for filling the sensor cavity, the surface of the sensor cavity is closed by means of a membrane, e.g., made of a polymer or a metal such as titanium. No surface treatment of the sensor cavity is necessary when using a solid and/or gel-like silicone. However, due to a possibly occurring diffusion of water into the silicone, the silicone can swell and thereby, for example, cause a tension on the pressure sensor membrane of the pressure sensor, which in turn can lead to an error of the pressure sensor. The solid and/or gel-like silicone can therefore be coated with a water barrier and/or diffusion barrier on a parylene basis, for example.

The approach presented here furthermore creates a heart support system with a sensor head device, wherein the sensor head device is arranged at one end of a guide cannula, wherein the sensor head device is in particular electrically connectable or electrically connected by means of a connecting element to an electrical conductive element of the guide cannula. The special advantages of the approach presented here can also be realized easily and cost-effectively by such an embodiment.

A method for producing a sensor head device for a heart support system is presented, wherein the method comprises at least the following step:
forming a sensor carrying element having at least one sensor cavity for accommodating at least one sensor and/or at least one signal transmitter cavity for accommodating at least one signal transmitter in order to produce a sensor head device for a heart support system.

According to one embodiment, the method can comprise a step of inserting at least one sensor into the sensor cavity and/or at least one signal transmitter into the at least one signal transmitter cavity of the shaped sensor carrying element.

The method presented here for producing a sensor head device for a heart support system can be implemented, for example, in software or hardware or in a mixed form of software and hardware in a control device, for example.

The approach presented here furthermore creates a device that is designed to carry out, control, or implement in corresponding devices the steps of a variant of a method presented here for producing a sensor head device for a heart support system. The object underlying the invention can also be achieved quickly and efficiently with this design variant of the invention in the form of a device.

For this purpose, the device can have at least one computing unit for processing signals or data, at least one memory unit for storing signals or data, at least one interface to a sensor or an actuator for reading sensor signals from the sensor or for outputting data or control signals to the actuator and/or at least one communication interface for reading or outputting data that are embedded in a communication protocol. The computing unit can, for example, be a signal processor, a microcontroller, or the like, whereby the memory unit can be a flash memory, an EEPROM, or a magnetic memory unit. The communication interface can be designed to read or output data in a wireless and/or wire-bound manner, wherein a communication interface that can read or output wire-bound data can, for example, read said data electrically or optically from a corresponding data transmission line or output them into a corresponding data transmission line.

In the present case, a device can be understood to mean an electrical device that processes sensor signals and, as a function thereof, outputs control signals and/or data signals. The device can have an interface, which can be designed by means of hardware and/or software. In a design by means of hardware, the interfaces can, for example, be part of a so-called system ASIC that includes various functions of the device. However, it is also possible that the interfaces are separate, integrated circuits or at least partially consist of discrete structural elements. In a design by means of software, the interfaces can be software modules, which are present on a microcontroller in addition to other software modules, for example.

A computer program product or computer program having program code which can be stored on a machine-readable carrier or storage medium such as a semiconductor memory, a hard drive memory, or optical memory and is used to carry out, implement, and/or control the steps of the method according to one of the embodiments described above is also advantageous, in particular if the program product or program is executed on a computer or a device.

### BRIEF DESCRIPTION OF THE DRAWINGS

Exemplary embodiments of the approach presented here are shown schematically in the drawings and explained in more detail in the following description. The drawings show:

FIG. 1 a schematic view of a heart support system according to an exemplary embodiment;

FIG. 2 a schematic view of a first design variant of a sensor head device for a heart support system according to an exemplary embodiment;

FIG. 3A a schematic view of a second design variant of a sensor head device for a heart support system according to an exemplary embodiment;

FIG. 3B a schematic view of a further design variant of a sensor head device for a heart support system according to an exemplary embodiment;

FIG. 3C a schematic view of a further design variant of a sensor head device for a heart support system according to an exemplary embodiment;

FIG. 4 a schematic view of a sensor carrying part of a sensor head device according to an exemplary embodiment;

FIG. 5 a schematic view of a cylindrical ultrasound element of a sensor head device according to an exemplary embodiment;

FIG. 6 a flow diagram of an exemplary embodiment of a method for producing a sensor head device for a heart support system according to an exemplary embodiment; and

FIG. 7 a block diagram of a device for producing a sensor head device for a heart support system according to an exemplary embodiment.

In the following description of favorable exemplary embodiments of the present invention, the same or similar reference signs are used for the elements which are shown in the various figures and have a similar effect, wherein a repeated description of these elements is omitted.

### DETAILED DESCRIPTION

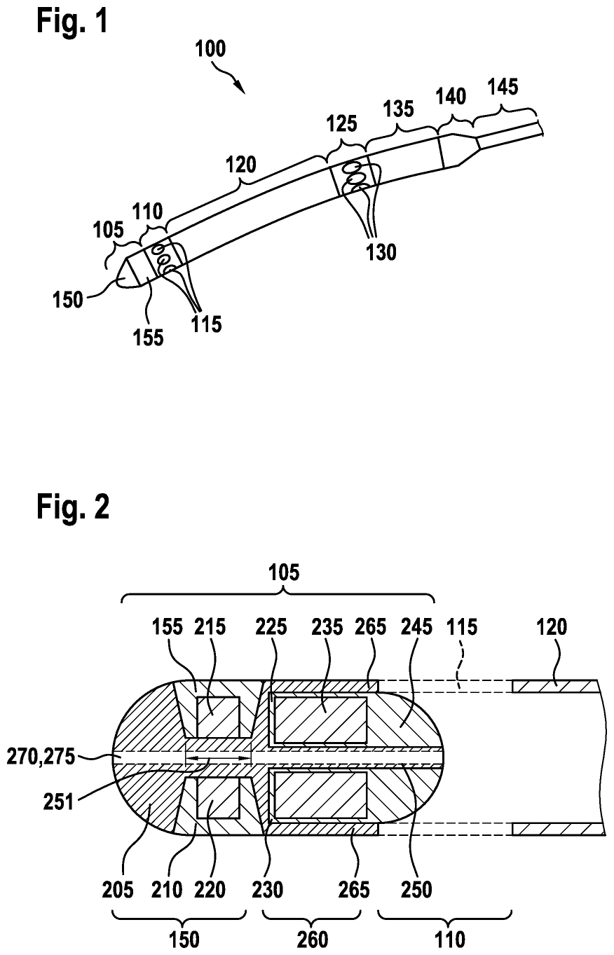

FIG. 1 shows a schematic view of a heart support system 100 according to an exemplary embodiment. By way of example, FIG. 1 shows a left ventricular heart support system 100 with an integrated sensor head device 105. The heart support system 100 has, by way of example, a cylindrical, elongated structure with a substantially constant outer diameter and rounded, tapered ends for easy positioning by means of a catheter in a blood vessel, e.g., the left heart chamber or the aorta.

The heart support system 100, here by way of example a left ventricular heart support system 100 for percutaneous implantation into a left heart chamber, first has the sensor head device 105. The heart support system 100 furthermore has an inlet cage 110 with a plurality of inlet openings 115 via which the blood of the heart disease patient enters the heart support system 100, a guide cannula 120, an impeller cage 125 with impeller and a plurality of outlet openings 130, a coupling and motor housing 135, a back end 140 with sensor technology, optional sensor hub, and an electrical connection field, as well as lastly a connection cable 145 for connecting the heart support system 100 to an external energy source or an external evaluation or control device.

The sensor head device 105 of the heart support system 100 has, by way of example, a rounded outer end 150 facing away from the heart support system 100 as well as a sensor assembly which is arranged in at least one sensor cavity 155, which may also be designed as a circumferential groove, of the sensor head device 105 and which is used, for example, to measure the pressure and/or temperature of a heart disease patient. In this case, the sensor head device 105 is arranged at an inner end (not shown) on the inlet cage 110, wherein the inlet cage 110 can also be designed as an integral component of the guide cannula 120. In addition, the sensor head device 105 can, for example, be electrically connected by means of a connecting element (not shown) of the inlet cage 110 to an electrical conductive element (not shown) of the guide cannula 120.

FIG. 2 shows a schematic view of a first design variant of a sensor head device 105 for a heart support system according to an exemplary embodiment. The sensor head device 105 is arranged by way of example at an inner end on the inlet cage 110 or the guide cannula 120, wherein the sensor head device 105 is electrically connected by means of a connecting element (not shown) of the inlet cage 110 to an electrical conductive element (not shown) of the guide cannula 120.

The sensor head device 105 has a sensor carrying element 205, which by way of example forms the base body of the sensor head device 105. The sensor carrying element 205 is, for example, made of a thermoplastic in an injection molding process but may alternatively also be manufactured in a machining manufacturing process.

The sensor carrying element 205 shown here has, by way of example, a circumferential or at least partially circumferential sensor cavity (155 and 210) (which, however, may also be designed as two separate sensor cavities 155 and 210) for accommodating by way of example two sensors 215 and 220. The sensor carrying element 205 furthermore has, by way of example, a cylindrical signal transmitter cavity (225 and 230) for accommodating a cylindrical signal transmitter 235. The sensor 215 and/or 220 is, by way of example, a temperature sensor and/or pressure sensor but in particular a barometric absolute pressure sensor. The signal transmitter 235 is, by way of example, an ultrasound element, onto which, according to one exemplary embodiment, a lens element 245, e.g., an ultrasonic lens, made of silicone is positioned, wherein the lens element 245 at least partially covers the signal transmitter cavity 225.

According to one exemplary embodiment, the signal transmitter cavity 225 is open in a different direction than the sensor cavity 155, wherein the signal transmitter cavity 225 is open in a direction facing the heart support system so that the main beam direction of the signal transmitter points into the axis of the guide cannula 120. The at least one sensor cavity 155 is manufactured by way of example as an at least partially circumferential groove (e.g., at an angle of 330° in the circumferential direction around an outer surface of the sensor head device 105) and is radially open. In a particularly advantageous manner, the walls of the groove as a sensor cavity 155 are designed in such a way that the groove is wider toward the outside than at the base. As a result, swelling of silicone, which is introduced in a production step of the sensor carrying element 205 as a seal into the groove as well as into a sensor 215 arranged therein, then leads less to a tension of the MEMS element as sensor 215.

The sensor carrying element 205 has a channel 270 extending along a longitudinal direction of extension of the sensor head device 105, wherein in the first design variant of a sensor carrying element 205 shown here, the channel 270 ends centrally, by way of example, in an outer end 150 of a rounded cap or a mushroom head form facing away from the heart support system. The mushroom head-shaped outer end 150 of the sensor head device 105 is rounded in order to not induce any injuries in the heart disease patient and to ensure good sliding properties when advancing the heart support system during the implantation of the system.

In its center, by way of example, the sensor carrying part 205 has an opening 271. In this case, the opening 271 serves to accommodate a guide wire (not shown). When implanting a heart support system into the left heart chamber or into the aorta of a heart disease patient, the guide wire is first laid to the ventricle of the patient. The heart support system is then pushed onto the guide wire and advanced along the guide wire to the end position.

In a first embodiment for guiding the guide wire in the region of the signal transmitter cavity, the sensor carrier 205 can include a tubular continuation 250, which extends so far into the signal transmitter cavity that the guide wire is guided to the contact surface between ultrasonic lens 245 and blood in the region 110. In a second embodiment, a small tube 275 (e.g., a metallic tube made of stainless steel, titanium, or NiTiNol, for example) is inserted into the channel 270 and extends through the sensor carrying part 205 and beyond it into the signal transmitter cavity 225 to the contact surface between ultrasonic lens 245 and blood in the region 110. The second embodiment has the advantage of a wall thickness that can be significantly less, which results in a reduced central opening width in the signal transmitter, which is advantageous above all when the signal transmitter is designed as an ultrasound transducer.

Toward the outside, the signal transmitter cavity is limited by a cylinder jacket 265, which is, by way of example, part of the design of the sensor carrying part 205. The cylindrical signal transmitter 235 can consequently be designed to have its central opening on the continuation 250 or the small tube 275 (depending on the design variant) and be inserted into the signal transmitter cavity 225. The gap between 250 and 235 or 275 and 235 as well as between 235 and 265 can be filled, for example, by epoxy or silicone, which also serves as adhesive fixation at the same time.

The mushroom head-shaped outer end 150 represents an additional mechanical protection for the sensors 215, 220 arranged in the at least one sensor cavity 155. In order to lay the sensors 215 and 220 around the web 251 (which is, for example, cuboidal here), it is necessary in the first design variant of a sensor head device 105 shown here to arrange the sensors 215 and 220 on a flexible circuit board or a thin-film substrate. The sensors 215, 220 are bonded to a thin-film substrate, for example. In the region of the sensors 215, 220, the thin-film substrate should not be bent, which is why it may be supported here with stiffening elements, but above all, the substrate is not cylindrically round but is designed as a cuboid with rounded corners. The rounded corners of the web 251 serve, for example, to maintain the bending radii of the thin-film substrate (polyimide gold layer structure).

According to one exemplary embodiment, the at least one sensor cavity 155 is filled with a casting compound to protect the sensors 215, 220 from blood and mechanical damage. In this case, the casting compound can be a solid and/or gel-like silicone or even a silicone oil. In this case, the casting compound should be able to transfer a cardiac pressure of a patient in order to continue to enable precise pressure measurement by the sensors 215 or 220.

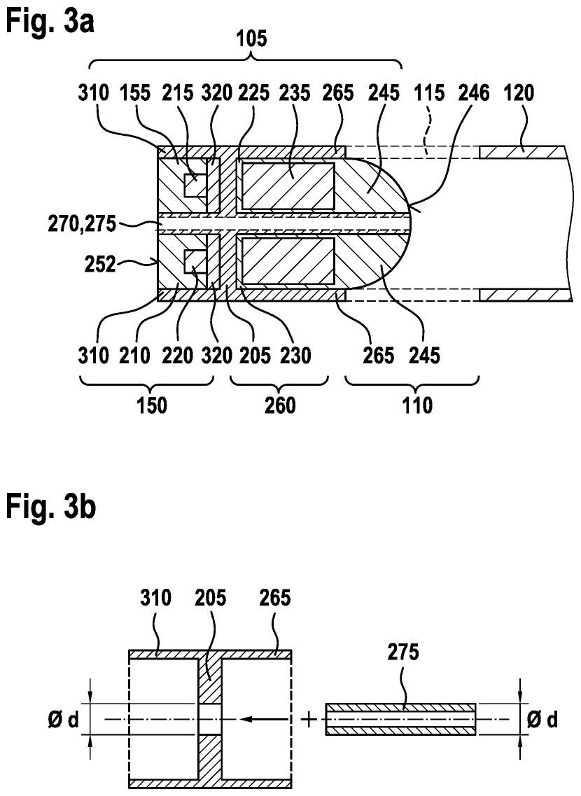

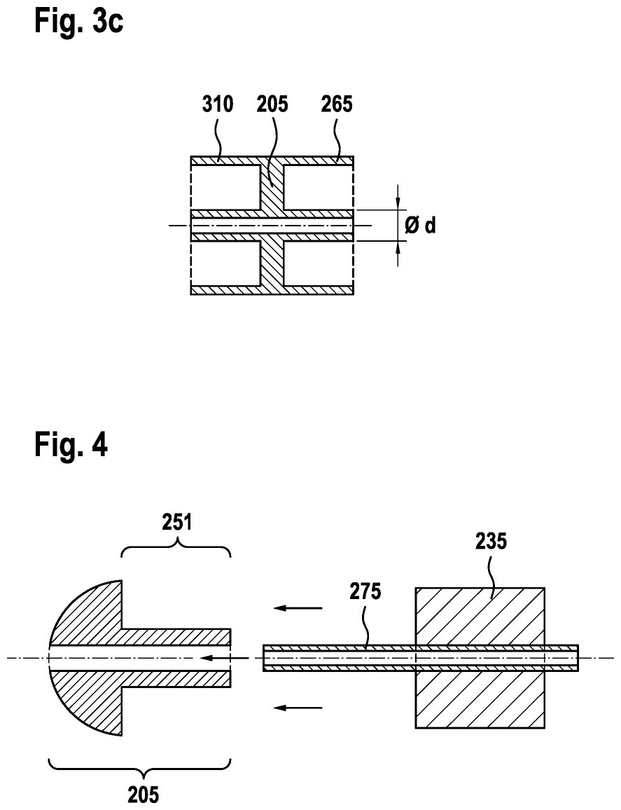

FIG. 3A shows a schematic view of a second design variant of a sensor head device 105 for a heart support system 100 according to an exemplary embodiment. The sensor head device 105 is arranged by way of example at an inner end on the guide cannula 120 or on the inlet cage 110, wherein the sensor head device 105 is electrically connected by means of a connecting element (which is not shown here) of the inlet cage 110 to an electrical conductive element (not shown) of the guide cannula 120. In the narrower sense, the electrical conductive element is, for example, laid completely in one piece from the back end 140 to the sensor head device 105 by affixing across all components of the sensor head device 205.

The sensor head device 105 has a sensor carrying element 205, which by way of example forms the base body of the sensor head device 105. The sensor carrying element 205 is, for example, made of a thermoplastic in an injection molding process but may alternatively also be manufactured in a machining manufacturing process.

The sensor carrying element 205 shown here has, by way of example, two sensor cavities 155 and 210 or, as already described in detail above, an at least partially circumferential groove as sensor cavity 155 for accommodating one sensor 215 or 220 each. The sensor carrying element 205 furthermore has, by way of example, a signal transmitter cavity 225 for accommodating a signal transmitter 235. The sensor 215 and/or 220 is, by way of example, a temperature sensor and/or pressure sensor but in particular a barometric absolute pressure sensor. The signal transmitter 235 is, by way of example, an ultrasound element, onto which, according to one exemplary embodiment, a lens element 245, e.g., an ultrasonic lens, made of silicone is positioned, wherein the lens element 245 at least partially covers the signal transmitter cavity 225 and 230.

According to one exemplary embodiment, the signal transmitter cavity 225 is open in a different direction than the sensor cavity 155 or the two sensor cavities 155 and 210 if the sensor cavity 155 is not formed by an at least partially circumferential groove, wherein the signal transmitter cavity 225 or the two signal transmitter cavities 225 or 230 are open in a direction facing the heart support system. The first sensor cavity 155 is open, by way of example, in a direction facing away from the heart support system, wherein the sensor cavity 155 is therefore open in a direction offset by 90° with respect to the signal transmitter cavity 225.

The sensor carrying element 205 has a channel 270 extending along a longitudinal direction of extension of the sensor head device 105, wherein the channel in the first design variant of a sensor head device 105 shown here extends through a web-like continuation 250 to the surface 252 of the sensor cavity or the surface of the signal transmitter 235 or the surface 246 of the optional ultrasonic lens. In an embodiment not shown, the web-like continuation 250 can be formed by a small tube 275 (e.g., a small metallic tube) so that the channel 270 can be designed with thin walls. The sensor cavity 155 located by way of example at an outer end 150 facing away from the heart support system can have an outer wall with a U-shaped cross section or, when using a web-shaped continuation 250, an E-shaped outer wall 310. According to one exemplary embodiment, the sensor cavity 155 or one sensor cavity 155, 210 each for accommodating a sensor 215, 220 is formed between an outer wall 310 each and the web-like continuation 250 or the small tube 275. This design variant of an outer end 150 thus has a pot shape by way of example. The continuation of the channel 270 to an inner end 260 can also be carried out by a web 250 or as a small tube 275. The signal transmitter cavity located there has walls with a U-shaped cross section or, when using the web 250, E-shaped walls, wherein the signal transmitter cavity 225 for accommodating a signal transmitter 235 is formed between a pair each of inner walls 265 according to one exemplary embodiment.

An advantage of the design variant of the sensor head device 105 shown here is that the sensors 215 and 220 can be pre-mounted, for example, on a circular circuit board 320, wherein in an alternative exemplary embodiment, the signal transmitter 235 can also be pre-mounted on a circular circuit board.

According to one exemplary embodiment, the sensor cavities 155 or the two sensor cavities 155, 210 are filled with a casting compound to protect the sensors 215, 220 from blood and mechanical damage. In this case, the casting compound can be a solid and/or gel-like silicone or even a silicone oil. In this case, the casting compound should be able to transfer the cardiac pressure of a patient in order to continue to enable precise pressure measurement by the sensors 155 and 210.

In its center, by way of example, the sensor carrying element 205 has a channel 270, wherein a cylindrical tube 275 is positioned in the channel 270 by way of example. The tube 275 (which can also be referred to as a small tube) can be inserted into and/or formed in the opening 271 over the entire length of the sensor head device 105. In this case, the opening 271 or the cylindrical tube 275 serves to accommodate a guide wire (not shown). When implanting a heart support system into the left heart chamber or into the aorta of a heart disease patient, the guide wire is first laid to the ventricle of the patient. The heart support system is then pushed onto the guide wire and advanced along the guide wire to the end position.

FIG. 3B shows a schematic view of a further design variant of a sensor head device 105 for a heart support system 100 according to an exemplary embodiment. In contrast to the design variant of the sensor head device 105 shown in FIG. 3A, the small cylindrical tube 275, which is inserted into the central part of the sensor carrying element 205, is now provided in the design variant shown in FIG. 3B in order to form the channel 270 or the opening 271. In this case, the small tube 275 has a diameter that is larger than the diameter of an opening of the sensor carrying element 205 so that after insertion into the sensor carrying element 205, the small tube 275 can be held in the sensor carrying element 205 by means of a press fit.

FIG. 3C shows a schematic view of a further design variant of a sensor head device for a heart support system according to an exemplary embodiment. The sensor carrying element 205 shown in FIG. 3C is shown here as a one-part or one-piece component, as used in the illustration of FIG. 3A, for example.

FIG. 4 shows a schematic view of a sensor carrying part 205 of a sensor head device 105 according to an exemplary embodiment. By way of example, the sensor carrying part 205 is realized in several parts in the illustration shown here. A simple demolding of the sensor carrying part 205 as an injection molded part made of a thermoplastic, e.g., of polyether ether ketone, is thus possible. In this case, the injection molded part or the sensor carrying element 205 comprises only the mushroom cap-shaped outer end 150 with the web 251, wherein a cylindrical tube 275, which is, for example, made of a metal material, is inserted into a centrally formed channel 270 of the sensor carrying part 205. The tube 275 can be inserted, by way of example, into the channel 270 over the entire length of the sensor carrying part 205 and serves, by way of example, to accommodate a guide wire (not shown). According to one exemplary embodiment, a sensor cavity 155 or one sensor cavity 155 and 210 each is formed on the side facing away from the mushroom cap above and below the web 251, wherein said sensor cavity 155 (or the sensor cavities 155 and 210 each) serve to accommodate at least one sensor. The individual parts of the sensor carrying part 205 can, by way of example, be joined together by means of a biocompatible adhesive.

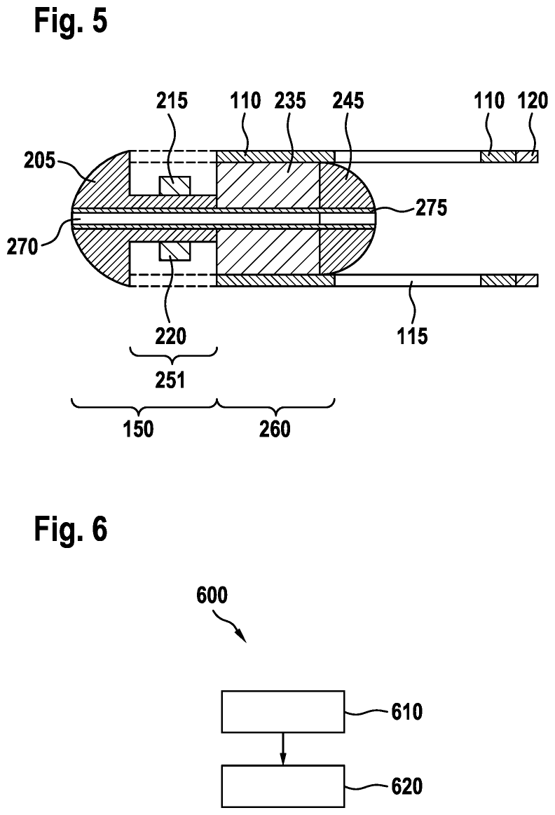

FIG. 5 shows a schematic view of a cylindrical ultrasound element 235, which is pushed onto a sensor carrying element 205 of a sensor head device 105 according to an exemplary embodiment. By way of example, the ultrasound element 235 shown here has an opening provided for pushing onto the tube 275. In this case, the tube 275 is inserted into the opening of the sensor carrying element in the region of the ultrasound element 235 in order to ensure encapsulation of the ultrasound element 235 from the inside as well. The ultrasound element 235 may, by way of example, be piezoelectric elements with necessary backing and impedance adaptation layers and/or may be hermetically sealed elements. The ultrasound element 235 is arranged, by way of example, on a circular circuit board and in this case integrated into the sensor head device and encapsulated therein such that the ultrasound element 235 points in the direction of the guide cannula. The ultrasound element 235 is thus optimally integrated into and securely encapsulated in the cylindrical shape of the guide cannula or of the entire heart support system.

FIG. 6 shows a flow chart of an exemplary embodiment of a method 600 for producing a sensor head device for a heart support system according to an exemplary embodiment.

In a step 610 of the method 600, a sensor carrying element with at least one sensor cavity for accommodating at least one sensor and/or at least one signal transmitter cavity for accommodating at least one signal transmitter is formed in order to produce a sensor head device for a heart support system. In a step 620 of the method 600, lastly, at least one sensor is inserted into the sensor cavity and/or at least one signal transmitter is inserted into the at least one signal transmitter cavity of the formed sensor carrying element.

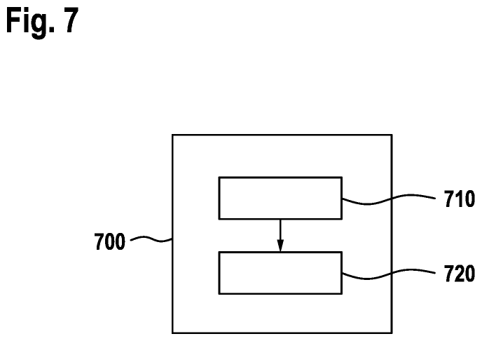

FIG. 7 shows a block diagram of a device 700 for producing a sensor head device for a heart support system according to an exemplary embodiment. In this case, the device 700 is designed to carry out and/or control the steps of a method for producing a sensor head device in corresponding units.

The device 700 thus has a forming unit 710 as well as an insertion unit 720. The forming unit 710 is designed in this case to form a sensor carrying element with at least one sensor cavity for accommodating at least one sensor and/or at least one signal transmitter cavity for accommodating at least one signal transmitter in order to produce a sensor head device for a heart support system. The insertion unit 720 is lastly designed to insert at least one sensor into the at least one sensor cavity and/or at least one signal transmitter into the at least one signal transmitter cavity of the formed sensor carrying element.

If an exemplary embodiment includes an “and/or” conjunction between a first feature and a second feature, this should be read to mean that the exemplary embodiment according to one embodiment comprises both the first feature and the second feature and according to another embodiment comprises either only the first feature or only the second feature.

What is claimed is:

1. A heart support system comprising:
a heart pump comprising:
a guide cannula; and
an inlet opening in a side wall of the guide cannula; and
a sensor head device comprising:
at least one signal transmitter cavity configured to receive at least one signal transmitter; and
a tapered cap at an outer end;
wherein the sensor head device is positioned distal to a first end of the guide cannula and the at least one signal transmitter cavity is distal to the inlet opening in the side wall of the guide cannula.

2. The heart support system according to claim 1, wherein the sensor head device is configured to electrically connect to a back end of the heart support system positioned proximal to a second end of the guide cannula.

3. The heart support system of claim 1, wherein the at least one signal transmitter cavity is open in a direction of the guide cannula.

4. The heart support system of claim 1, further comprising at least one signal transmitter.

5. The heart support system of claim 4, wherein the at least one signal transmitter comprises a signal transducer.

6. The heart support system of claim 5, wherein the signal transducer comprises an ultrasound transducer.

7. The heart support system according to claim 4, wherein the at least one signal transmitter is arranged on an electrical conductive element.

8. The heart support system of claim 4, wherein the at least one signal transmitter cavity is at least partially covered by a lens element, wherein the lens element comprises an ultrasonic lens and/or a lens made of a silicone material.

9. The heart support system according to claim 4, wherein the at least one signal transmitter cavity comprises a cylinder jacket which forms an outer surface of the at least one signal transmitter cavity.

10. The heart support system according to claim 8, wherein the at least one signal transmitter is cylindrical.

11. The heart support system according to claim 9, further comprising a tube extending through a central opening in the at least one signal transmitter.

12. A method for producing a sensor head device for a heart support system, comprising:
forming a sensor carrying element configured to be delivered to a heart via a catheter, the sensor carrying element comprising:
at least one signal transmitter cavity configured to receive at least one signal transmitter; and
a tapered cap at an outer end;
wherein the sensor head device is configured to attach to a heart pump, the heart pump comprising:
a guide cannula; and
an inlet opening in a side wall of the guide cannula;
wherein the sensor head device is configured to be positioned distal to a first end of the guide cannula and the at least one signal transmitter cavity are distal to the inlet opening in the side wall of the guide cannula.

13. The method of claim 12, further comprising inserting at least one signal transmitter into the at least one signal transmitter cavity of the formed sensor carrying element.

14. The method of claim 13, wherein the at least one signal transmitter comprises a signal transducer.

15. The method of claim 14, wherein the signal transducer comprises an ultrasound transducer.

16. The method of claim 13, further comprising inserting a tube through a central opening of the at least one signal transmitter.

17. The method of claim 13, wherein the at least one signal transmitter is arranged on an electrical conductive element.

18. The method of claim 13, further comprising electrically connecting the sensor head device to a back end of the heart support system positioned proximal to a second end of the guide cannula.

19. The method of claim 12, wherein the at least one signal transmitter cavity is open in a direction of the guide cannula.

20. The method of claim 12, further comprising at least partially covering the at least one signal transmitter cavity by a lens element, wherein the lens element comprises an ultrasonic lens and/or a lens made of a silicone material.
